(19) United States
(12) Patent Application Publication
(10) Pub. No.: US 2025/0009495 A1
(43) Pub. Date: Jan. 9, 2025

(54) BILIARY STENT AND DELIVERY DEVICE

(71) Applicant: Boston Scientific Scimed, Inc., Maple Grove, MN (US)

(72) Inventor: Martyn G. Folan, Loughrea (IE)

(73) Assignee: Boston Scientific Scimed, Inc., Maple Grove, MN (US)

(21) Appl. No.: 18/763,809

(22) Filed: Jul. 3, 2024

Related U.S. Application Data
(60) Provisional application No. 63/512,554, filed on Jul. 7, 2023.

(51) Int. Cl. A61F 2/04; A61F 2/966

(52) U.S. Cl. CPC A61F 2/04; A61F 2/966; A61F 2002/041

(57) ABSTRACT

Devices, systems, and methods for deploying a biliary stent. A biliary stent has lobes and creases to expand between contracted and expanded positions. The stent is delivered to a bile duct in a contracted position loaded on a shaft of a delivery device. When the shaft is retracted through the stent, wings on the shaft force the stent outward into the larger expanded position. In the expanded position, the creases form drainage channels to reduce blockage over the life of the stent.

### CROSS REFERENCE TO RELATED APPLICATIONS

This application claims the benefit of U.S. Provisional Patent Application Ser. No. 63/512,554 filed on Jul. 7, 2023, the disclosure of which is incorporated herein by reference.

### TECHNICAL FIELD

This disclosure relates generally to medical devices and, more particularly, to expandable biliary stents.

### BACKGROUND

A partial or total blockage of one or more bile ducts may be remedied by use of biliary stents. Conventional biliary stents are tubes made of plastic or metal, generally having a circular or ovular cross-section. With the advent of endoscopic surgery to install the stents, the maximum radius of each stent is limited by the maximum diameter in the endoscopic device channel used to deliver them. Expandable biliary stents are known, but the stent's smooth cylindrical shape can result in the stent filling the bile duct and quickly becoming clogged. There is a need for a reliable method for delivering and deploying an expandable stent with easy, mechanical expansion and exterior channels for drainage.

The present disclosure provides medical devices and methods of using the same that avoid the aforementioned shortcomings of existing devices.

### SUMMARY

This disclosure provides design, material, manufacturing method, and use alternatives for medical devices and medical systems. In a first example, a biliary stent comprises a plurality of lobes alternating radially with a plurality of creases; wherein each of the plurality of lobes has a first contracted position biased inwards from the creases such that the biliary stent has a first cross-sectional diameter; and wherein each of the plurality of lobes has a second expanded position biased outwards from the creases such that the biliary stent has a second cross-sectional diameter larger than the first cross-sectional diameter.

Alternatively or additionally to any of the examples above, the plurality of lobes are the same length such that the stent is symmetrical.

Alternatively or additionally to any of the examples above, the stent is made of biocompatible plastic.

Alternatively or additionally to any of the examples above, each of the lobes forms a convex curve with adjacent creases when in the second expanded position such that adjacent lobes form side walls of channels exterior to the stent upon expansion of the biliary stent.

Alternatively or additionally to any of the examples above, the stent in the first contracted position has a cross-sectional diameter of between 1 mm and 5 mm.

Alternatively or additionally to any of the examples above, the stent in the second expanded position has a cross-sectional diameter of between 2 mm and 10 mm.

Alternatively or additionally to any of the examples above, the stent has an axial length of between 2 mm and 20 mm.

In another example, a biliary stent delivery device comprises a shaft sized for insertion into a bile duct of a patient and one or more wings at the distal end of the shaft, wherein each wing shifts between a first proximal orientation pointed towards a proximal end of the shaft and a second distal orientation pointed towards the distal end of the shaft.

Alternatively or additionally to any of the examples above, the shaft is loaded with an expandable biliary stent for insertion into the bile duct, the expandable stent positioned proximally to the one or more wings such that, when the shaft is retracted through the loaded stent, the wings shift from the first proximal orientation to the second distal orientation to press outward on the stent, expanding the stent.

Alternatively or additionally to any of the examples above, the wings have a cross-sectional diameter in the first proximal orientation of between 2.3 mm and 4.2 mm.

Alternatively or additionally to any of the examples above, the wings have a cross-sectional diameter in the second proximal orientation of between 2.3 mm and 4.2 mm.

In another example, a method of deploying a biliary stent of any of the examples above can include the steps of: loading the stent in the first position onto the shaft of the delivery device of any one of the examples above; inserting the distal end of the shaft into the bile duct of a patient; positioning the stent within the patient; expanding the stent into the second position; and retracting the shaft, leaving the expanded stent in the bile duct.

Alternatively or additionally to any of the examples above, the one or more wings at the distal end of the shaft are positioned in the first proximal orientation during insertion of the distal end of the shaft and positioning the stent. Retracting the shaft includes moving the distal end of the shaft through the stent such that the wings shift from the first proximal orientation to the second distal orientation. Expanding the stent is a result of the wings pressing outward on the stent as the distal end of the shaft moves through the stent.

Alternatively or additionally to any of the examples above, the method further includes the step of deploying a second stent loaded onto the device

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a medical delivery device;

FIGS. 2A and 2B depict a biliary stent in a contracted position;

FIGS. 3A and 3B depicts the biliary stent in the expanded position;

FIG. 4 depicts the wings of a delivery device in a proximal orientation;

FIG. 5 depicts the wings in a distal orientation;

FIGS. 6A to 6E illustrate deployment of a biliary stent loaded on the shaft of a medical delivery device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a” “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

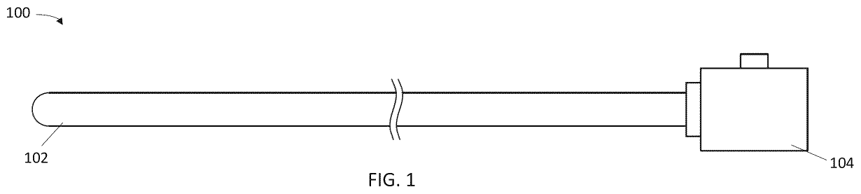

FIG. 1 shows a biliary access device 100 in accordance with some aspects of the present disclosure. The device 100 includes a distal shaft 102 suitable for insertion into or near the bile ducts of a patient. The shaft 102 is sized and shaped to be fed through an accessory channel of an endoscope via guidewire with one or more biliary stents loaded on the shaft 102. A proximal handle 104 includes any necessary interface components for controlling the distal shaft 102, for unloading the biliary stents, and for expanding the stents during deployment as further described herein. The handle 104 provides control of the shaft 102 during delivery and deployment of the stent and eventual retraction and removal of the device 100.

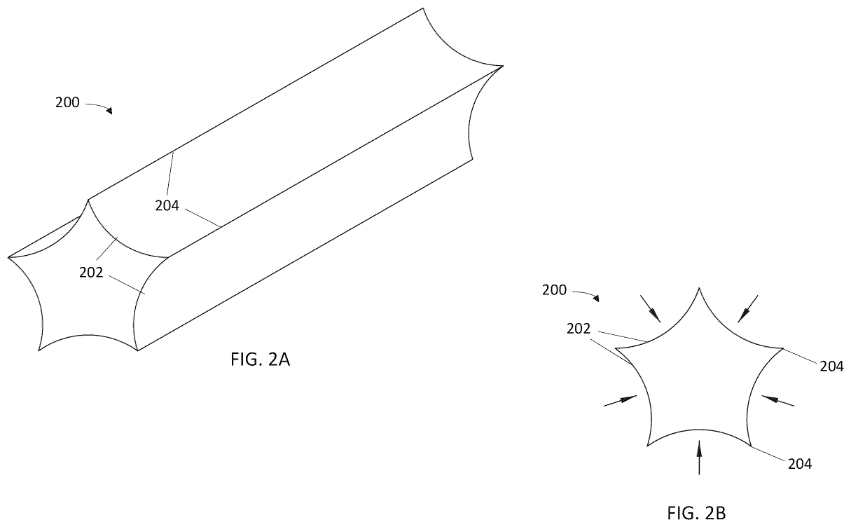

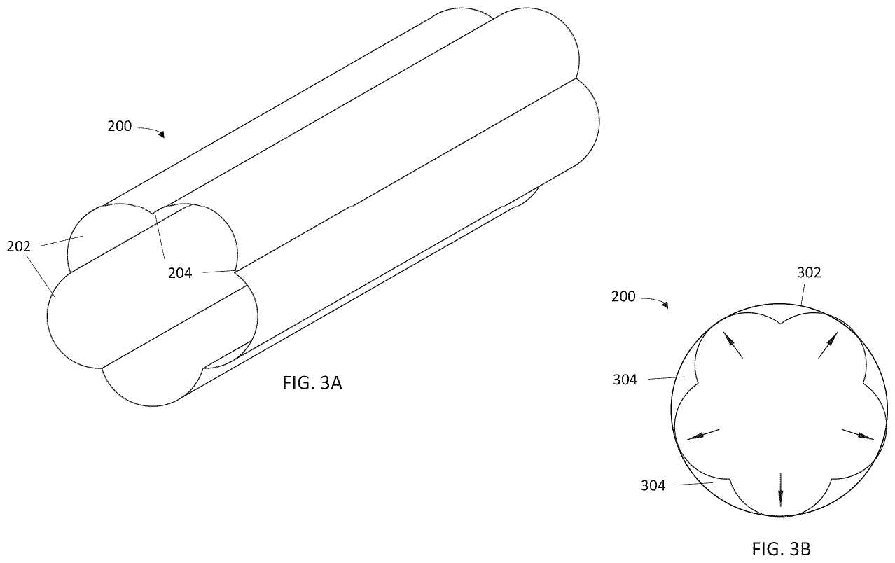

A biliary stent 200 is shown in FIGS. 2A-3B. The biliary stent 200 may have a first collapsed form in which a plurality of lobes 202 are biased concave inward between creases 204. The stent 200 may be formed, in some implementations, of a resilient material such as hard thermoplastic that maintains its shape. One of ordinary skill will recognize that any bio-compatible material may be used, but the appropriateness of the material may depend on the nature of the procedure as well as the placement of the stent. In FIGS. 3A and 3B, the lobes 202 are biased convex outward between the creases 204, greatly expanding the cross-sectional area of the stent 200. In this regard, the creases 204 act as hinged locations for the transition of the lobes 202 from the concave inward orientation to the convex outward orientation. By expanding after deployment as further described herein, the stent 200 can have an effective radius when deployed that is greater than the channel in which it was delivered.

The biliary stent 200 is illustrated with five creases and five lobes of symmetrical arc length, although one of ordinary skill will recognize that different numbers of lobes are possible, including three, four, six, seven, eight, etc., and that one or more lobes may be longer than one or more other lobes.

As shown in FIG. 3B, the expanded stent 200 creates an opening with the surrounding biliary duct tissue 302 that includes exterior channels 304. These channels 304 provide for drainage along the outside of the stent 200 and increase the useful life of the stent 200 over smooth symmetrical sidewalls conventionally found in plastic tube stents in the art.

The length and width of the stent 200 can depend on its intended placement and/or the limitations of the access device. In some implementations, the stent may have an axial length of between 2 and 20 mm and may expand in cross-sectional diameter from between 1 mm and 5 mm when contracted to between 2 mm and 10 mm when expanded.

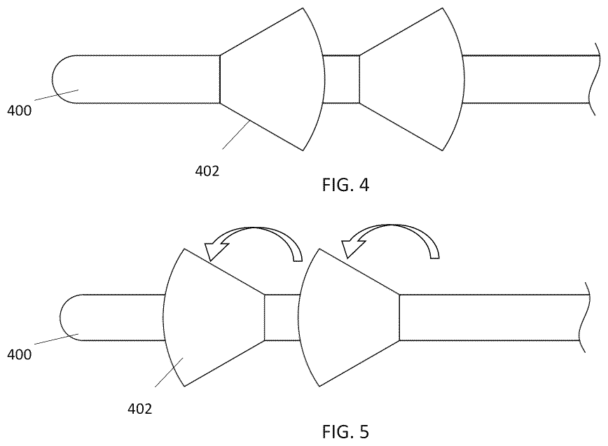

Deployment of the biliary stent is aided by a shaft 400 having wings 402 as shown in FIGS. 4 and 5, which can shift between a first position oriented proximally, as shown in FIG. 4, and a second position oriented distally, as shown in FIG. 5. The wings 402 may be made integrally with the shaft 400 or may be attached to the shaft 400, either mechanically or through adhesive.

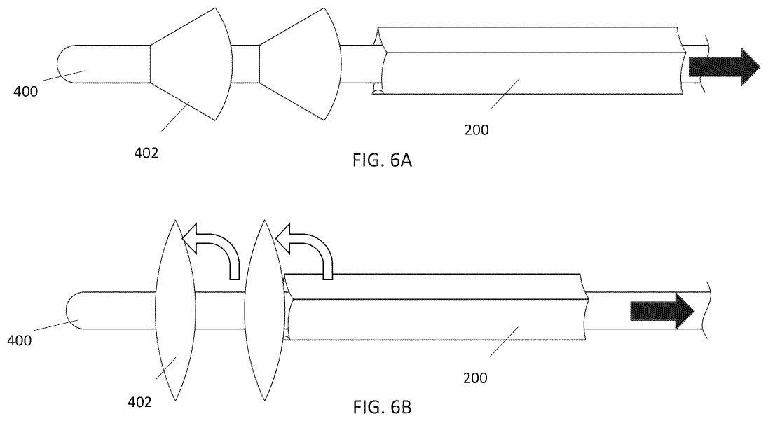

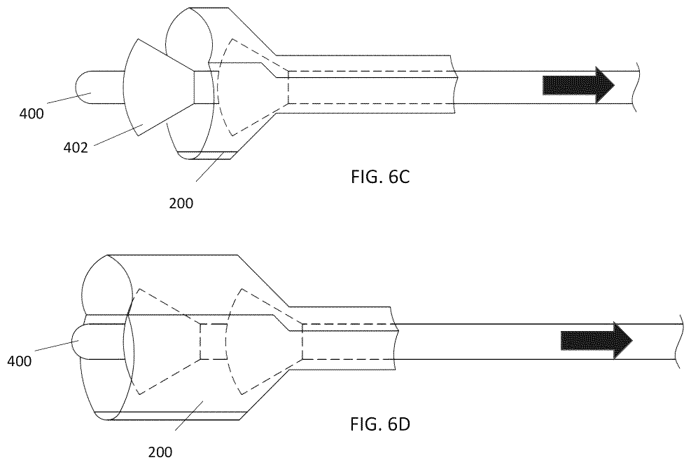

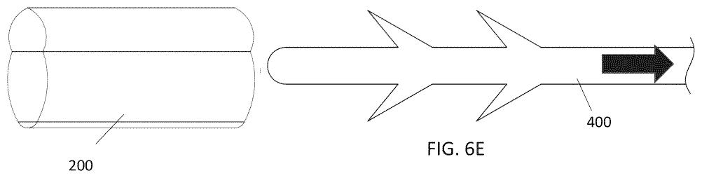

FIGS. 6A through 6E illustrate deployment of a biliary stent 200 over the shaft 400. As the shaft 400 retracts through the stent 200, the wings 402 are shifted from a proximal orientation to a distal orientation by contact with the stent 200, with the wings 402 passing through an intermediate orientation between the proximal orientation and the distal orientation. The wings 402, which have sufficient resilience and diameter not to collapse fully against the body of the shaft 400, push the stent 200 outward as they pass through, opening the stent 200 and causing the stent 200 to reorient from a collapsed state in which the lobes are in a concave inward orientation to an expanded state in which the lobes are in a convex outward orientation. Because the wings 402 are flexible and may tend to flatten due to the resistance offered by the collapsed stent 200, the use of multiple wings in series may be used in some embodiments to enhance the effectiveness of the leading wing, because the next wing in the series acts as a barrier and thus buttresses the leading wing's ability to overcome the collapsed stent resistance.

The cross-sectional diameter of the wings 402 relates to the cross-sectional diameter of the stent 200 both before and after expansion and may be the same or different when oriented in first or second positions. In some implementations, each of the first and second wing positions may have a cross-sectional diameter of between 2.3 mm and 4.2 mm.

In some implementations, the wings 402 may be biased to naturally return to a distal orientation against the shaft 400 once the stent 200 as been expanded and deployed. In some implementations, a mechanism such as a guide wire or string may be provided to fold the wings 402 against the shaft 400. Several stents 200 may be loaded upon one shaft 400 for deployment during a single operation.

The stent 200 may be made of any biocompatible material, such as a polyethylene plastic, with sufficient flexibility and resilience. The wings 402 likewise may be made of any material that allows the appropriate shifting between positions. In some implementations, the wings 402 may be made of a more rigid material than the stent 200 to assure the correct deployment interaction between the two.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A biliary stent comprising a plurality of lobes alternating radially with a plurality of creases;
wherein each of the plurality of lobes has a first contracted position biased inwards from the creases such that the biliary stent has a first cross-sectional diameter; and
wherein each of the plurality of lobes has a second expanded position biased outwards from the creases such that the biliary stent has a second cross-sectional diameter larger than the first cross-sectional diameter.

2. The biliary stent of claim 1, wherein the plurality of lobes are the same length such that the stent is symmetrical.

3. The biliary stent of claim 1, wherein each of the lobes forms a convex curve with adjacent creases when in the second expanded position such that adjacent lobes form side walls of channels exterior to the stent upon expansion of the biliary stent.

4. The biliary stent of claim 1, wherein the stent in the first proximal position has a cross-sectional diameter of between 1 mm and 5 mm.

5. The biliary stent of claim 1, wherein the stent in the second distal position has a cross-sectional diameter of between 2 mm and 10 mm.

6. The biliary stent of claim 1, wherein the stent has an axial length of between 2 mm and 20 mm.

7. A biliary stent delivery device, comprising:
a shaft sized for insertion into a bile duct of a patient; and
one or more wings at the distal end of the shaft, wherein each wing shifts between a first proximal orientation pointed towards a proximal end of the shaft and a second distal orientation pointed towards the distal end of the shaft.

8. The device of claim 7, wherein the shaft is loaded with an expandable biliary stent for insertion into the bile duct, the expandable stent positioned proximally to the one or more wings such that, when the shaft is retracted through the loaded stent, the wings shift from the first proximal orientation to the second distal orientation to press outward on the stent, expanding the stent.

9. A medical system for delivering a biliary stent, comprising:
a biliary stent delivery device having a shaft sized for insertion into a bile duct of a patient and one or more wings at the distal end of the shaft, wherein each wing shifts between a first proximal orientation pointed towards a proximal end of the shaft and a second distal orientation pointed towards the distal end of the shaft; and
a biliary stent comprising a plurality of lobes alternating radially with a plurality of creases, wherein each of the plurality of lobes has a first contracted position biased inwards from the creases such that the biliary stent has a first cross-sectional diameter, and wherein each of the plurality of lobes has a second expanded position biased outwards from the creases such that the biliary stent has a second cross-sectional diameter larger than the first cross-sectional diameter.

10. The system of claim 9, wherein the shaft is loaded with the biliary stent for insertion into the bile duct, the expandable stent positioned proximally to the one or more wings such that, when the shaft is retracted through the loaded stent, the wings shift from the first proximal orientation to the second distal orientation to press outward on the stent, expanding the stent from the first contracted position into the second expanded position.

11. The system of claim 9, wherein the plurality of lobes of the biliary stent are the same length such that the stent is symmetrical.

12. The system of claim 9, therein the biliary stent is comprised of biocompatible plastic.

13. The system of claim 9, wherein each of the lobes of the biliary stent forms a convex curve with adjacent creases when in the second expanded position such that adjacent lobes form side walls of channels exterior to the stent.

14. The system of claim 9, wherein the stent in the first proximal position has a cross-sectional diameter of between 1 mm and 5 mm.

15. The system of claim 9, wherein the stent in the second distal position has a cross-sectional diameter of between 2 mm and 10 mm.

16. The system of claim 9, wherein the wings have a cross-sectional diameter in the first proximal orientation of between 2.3 mm and 4.2 mm.

17. The system of claim 9, wherein the wings have a cross-sectional diameter in the second distal orientation of between 2.3 mm and 4.2 mm.

18. A method of operating the system of claim 9, comprising:
loading the biliary stent in the first position onto the shaft of the delivery device;
inserting the distal end of the shaft into the bile duct of a patient;
positioning the stent within the patient;
expanding the stent into the second position; and
retracting the shaft, leaving the expanded stent in the bile duct.

19. The method of claim 18, wherein the one or more wings at the distal end of the shaft are positioned in the first proximal orientation during insertion of the distal end of the shaft and positioning the stent; and
wherein retracting the shaft comprises moving the distal end of the shaft through the stent such that the wings shift from the first proximal orientation to the second distal orientation;
and wherein expanding the stent is a result of the wings pressing outward on the stent as the distal end of the shaft moves through the stent.

20. The method of claim 18, further comprising deploying a second stent loaded onto the device.
